(19) United States
(12) Patent Application Publication
(10) Pub. No.: US 2010/0069666 A1
(43) Pub. Date: Mar. 18, 2010

(54) IMPROVED METHOD FOR THE PRODUCTION OF UNSATURATED CARBOXYLIC ACID ANHYDRIDES

(75) Inventor: Dirk Broell, Langen (DE)

Correspondence Address: OBLON, SPIVAK, MCCLELLAND MAIER & NEUSTADT, L.L.P., 1940 DUKE STREET, ALEXANDRIA, VA, 22314, US

(73) Assignee: Evonik Roehm GmbH, Darmstadt (DE)

(21) Appl. No.: 12/517,563

(22) PCT Filed: Sep. 3, 2007
(86) PCT No.: PCT/EP2007/059157

§ 371 Date: Jun. 4, 2009

(30) Foreign Application Priority Data
Dec. 18, 2006 (DE) 10 2006 060 162.9

(51) Int. Cl. C07C 51/083

(52) U.S. Cl. 562/888

(57) ABSTRACT

The invention provides an improved process for preparing unsaturated carboxylic anhydrides of the general formula I

R—C(O)—O—C(O)—R (I)

in which R is an unsaturated organic radical having 2 to 12 carbon atoms
by transanhydridizing an aliphatic carboxylic anhydride with a carboxylic acid of the general formula II

R—COOH (II)

in which R is as defined above,
in an apparatus consisting of a reaction region and a rectification column, characterized in that
   a) the reactants are fed into a reaction region in stoichiometric ratios,
   b) the carboxylic acid formed as a by-product is removed from the reaction mixture,
   c) the carboxylic anhydride of the formula I is subsequently drawn off,
   d) the unconverted reactants are recycled into the reaction region,
   e) the reaction region comprises a heterogeneous catalyst and
   f) one or more polymerization inhibitors are added.

The invention relates to an improved process for preparing unsaturated carboxylic anhydrides, especially the reaction of an unsaturated carboxylic acid with a low molecular weight aliphatic carboxylic anhydride.

DE-A-3510035 describes a process for preparing unsaturated carboxylic anhydrides by acid-catalysed transanhydridization reaction of acetic anhydride with an unsaturated carboxylic acid in the middle part of a distillation column. The catalysts specified are homogeneous catalysts, preferably sulphuric acid, aliphatic or aromatic sulphonic acids or phosphoric acid. To achieve complete conversion, acetic anhydride is used in an excess of 0.1 to 0.5 mol per mole of carboxylic acid, in which case a mixture of acetic acid and acetic anhydride is obtained at the top of the column, i.e. pure acetic acid is not obtained.

Furthermore, the product is formed contaminated by the catalyst, which has to be removed later in a further process step.

U.S. Pat. No. 4,857,239 describes a process for preparing methacrylic anhydride, wherein the molar ratio of methacrylic acid to acetic anhydride is 2.1 to 3 and a polymerization inhibitor is added into the distillation column. A disadvantage is that the reactant used in excess is obtained unused. Moreover, no catalyst which accelerates the reaction is used.

US-A-2003/0018217 describes a process for preparing methacrylic anhydride, wherein the initial molar ratio of methacrylic acid to acetic anhydride is preferably 9 to 11. The acetic acid formed is removed immediately, and the reactor contents released are diluted with acetic anhydride. To prevent polymerization, inhibitors are added to the reactor and to the column. Many by-products are formed, and cannot be removed completely. Another by-product which occurs is the acetyl (meth)acrylate intermediate (mixed anhydride of acetic anhydride and (meth)acrylic anhydride). No catalyst is used here either.

It is an object of the invention to provide an improved process for preparing unsaturated carboxylic anhydrides, in which a stoichiometric excess of one of the reactants is avoided, the formation of secondary components is substantially suppressed and a full conversion of the reactants is achieved. Furthermore, polymerization should be substantially prevented in all regions and the space-time yield of the reaction should be increased.

The invention provides an improved process for preparing unsaturated carboxylic anhydrides of the general formula I

R—C(O)—O—C(O)—R (I)

in which R is an unsaturated organic radical having 2 to 12 carbon atoms
by transanhydridizing an aliphatic carboxylic anhydride with a carboxylic acid of the general formula II

R—COOH (II)

in which R is as defined above,
in an apparatus consisting of a reaction region and a rectification column, characterized in that
a) the reactants are fed into a reaction region in stoichiometric ratios,
b) the carboxylic acid formed as a by-product is removed from the reaction mixture,
c) the carboxylic anhydride of the formula I is subsequently drawn off,
d) the unconverted reactants are recycled into the reaction region,
e) the reaction region comprises a heterogeneous catalyst and
f) one or more polymerization inhibitors are added.

These technical features achieve full conversion of the reactants, a significant increase in the space-time yield and substantial prevention of polymerization in all regions. Moreover, catalyst removal in a further separating apparatus is unnecessary.

Carboxylic acids suitable for the process according to the invention have an unsaturated organic radical having 2 to 12, preferably 2 to 6, more preferably 2 to 4 carbon atoms. Suitable alkenyl groups are the vinyl, allyl, 2-methyl-2-propenyl, 2-butenyl, 2-pentenyl, 2-decenyl, 1-undecenyl and the 9,12-octadecadienyl group. Particular preference is given to the vinyl group and the allyl group.

The particularly preferred carboxylic acids include (meth)acrylic acids. The term (meth)acrylic acids is known in the technical field, and is understood to refer to not only acrylic acid and methacrylic acid but also derivatives of these acids. These derivatives include β-methylacrylic acid (butenoic acid, crotonic acid), α,β-dimethylacrylic acid, β-ethylacrylic acid, α-chloroacrylic acid, α-cyanoacrylic acid, 1-(trifluoromethyl)acrylic acid and β,β-dimethylacrylic acid. Preference is give to acrylic acid (propenoic acid) and methacrylic acid (2-methylpropenoic acid).

Suitable carboxylic anhydrides for the process according to the invention are likewise known in the technical field. Preferred compounds have the general formula III R′—C(O)—O—C(O)—R′ (III) in which R′ is a C₁- to C₄-alkyl radical. Preference is given to using acetic anhydride.

According to the invention, a stoichiometric ratio is understood to mean a molar ratio of 1.9 to 2.1:1 of carboxylic acid to acetic anhydride.

For the removal of the product streams according to the present invention, it is possible to use any rectification column which has between 5 to 50 separating stages. The number of separating stages is preferably 20 to 30. In the present invention, the number of separating stages refers to the number of trays in a tray column multiplied by the tray efficiency, or the number of theoretical plates in the case of a column with structured packing or a column with random packing.

Examples of a rectification column with trays include those such as bubble-cap trays, sieve trays, tunnel-cap trays, valve trays, slotted trays, slotted sieve trays, bubble-cap sieve trays, jet trays, centrifugal trays; examples of a rectification column with random packing include those such as Raschig rings, Lessing rings, Pall rings, Berl saddles, Intalox saddles; and examples of a rectification column with structured packings include those of the Mellapak type (Sulzer), Rombopak type (Kühni), Montz-Pak (Montz), and structured packings with catalyst pockets, for example Katapak (Sulzer).

A rectification column with combinations of regions of trays, of regions of random packings and/or of regions of structured packings can likewise be used.

Preference is given to using a rectification column with random packings and/or structured packings.

The rectification column can be produced from any material suitable therefor. These include stainless steel and inert materials.

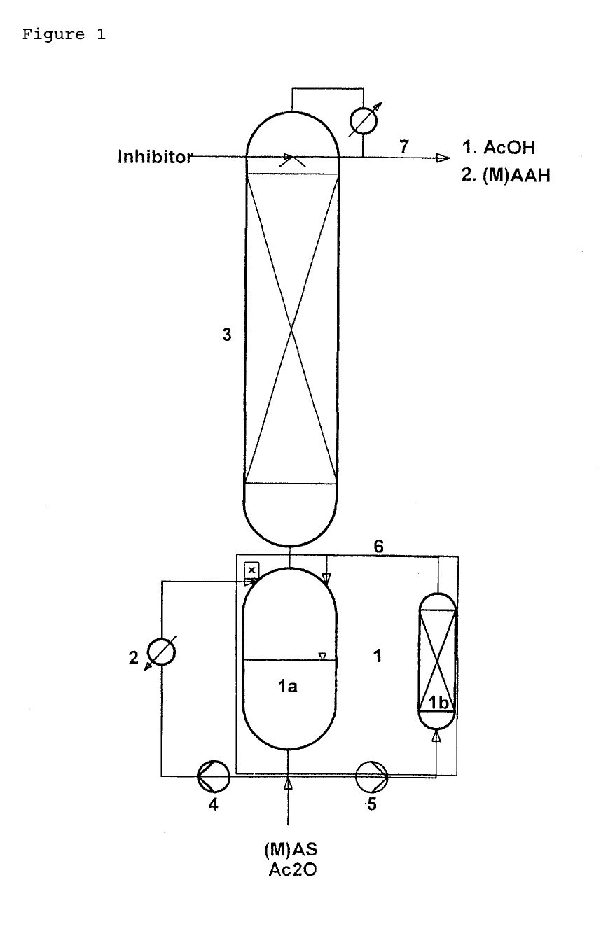

The apparatus has at least one region, referred to hereinafter as reaction region, in which at least one catalyst is preferably provided. This reaction region may be within and/or outside the rectification column. However, this reaction region is preferably arranged outside the rectification column, one of these preferred embodiment being shown in detail in FIG. 1.

The reaction is performed preferably at temperatures in the range of 30 to 120° C., more preferably at 40 to 100° C., especially at 50 to 80° C. The reaction temperature depends on the system pressure established. In the case of an arrangement of the reaction region within the column, the reaction is performed preferably within the pressure range of 5 to 100 mbar (absolute), especially at 10 to 50 mbar (absolute) and more preferably at 20 to 40 mbar (absolute).

If the reaction region is outside the column, different pressure and temperature conditions can be selected there from in the column. This has the advantage that the reaction parameters of the reactor can be established independently of the operating conditions in the column.

In the case of preparation of (meth)acrylic anhydride from acetic anhydride and (meth)acrylic acid, the reaction temperature is preferably 40 to 100° C., more preferably 50 to 90° C. and most preferably 70 to 85° C.

As well as the reactants, the reaction mixture may comprise further constituents, for example solvents, catalysts and polymerization inhibitors.

Preference is given to using heterogeneous catalysts in the reaction region. Particularly suitable heterogeneous catalysts are acidic fixed bed catalysts, especially acidic ion exchangers.

Surprisingly, it has been found with these acidic ion exchangers that the formation of the acetal (meth)acrylate intermediate (mixed anhydride of acetic anhydride and (meth)acrylic anhydride) is minimized and is present only in traces in the carboxylic anhydride of the formula I.

The particularly suitable acidic ion exchangers include especially cation exchange resins, such as sulphonic acid-containing styrene-divinylbenzene polymers. Suitable cation exchange resins can be obtained commercially from Röhm & Haas under the trade name Amberlyst®, from Dow under the trade name Dowex® and from Lanxess under the trade name Lewatit®.

The amount of catalyst in L is preferably 1/100 up to 1/1 of the reaction region, more preferably 1/30 to 1/5.

The polymerization inhibitors usable with preference include octadecyl 3-(3,5-di-tert-butyl-4-hydroxyphenyl)propionate, phenothiazine, hydroquinone, hydroquinone monomethyl ether, 4-hydroxy-2,2,6,6-tetramethylpiperidinooxyl (TEMPOL), 2,4-dimethyl-6-tert-butylphenol, 2,6-di-tert-butylphenol, 2,6-di-tert-butyl-4-methylphenol, para-substituted phenylene-diamines, for example N,N′-diphenyl-p-phenylenediamine, 1,4-benzoquinone, 2,6-di-tert-butyl-alpha-(dimethyl-amino)-p-cresol, 2,5-di-tert-butylhydroquinone or mixtures of two or more of these stabilizers. Very particular preference is given to phenothiazine.

The inhibitor can be metered into the feed upstream of the reaction region and/or into the rectification column, preferably at the top thereof.

According to the invention, the transanhydridization is effected in an apparatus in which the reactants are fed into the reaction region. The newly formed carboxylic acid, as the lowest-boiling reaction component, is drawn off at the top of the rectification column, preferably under reduced pressure.

Unconverted reactants and intermediates formed are recycled into the reaction region, for example by means of a pump.

Once the newly formed carboxylic acid has been drawn off completely, the unsaturated carboxylic anhydride of the formula I is subsequently drawn off at the top of the rectification column, preferably under reduced pressure.

The catalyst is provided in a separate part or in the entire reaction region of the apparatus. A separate arrangement of the catalyst is preferred, in which case unconverted reactants and intermediates formed are recycled into the part filled with catalyst. As a result, the unsaturated carboxylic anhydride, for example (meth)acrylic anhydride, and the newly formed carboxylic acid, for example acetic acid, are formed continuously.

A preferred embodiment of the process according to the invention is shown schematically in FIG. 1. The reaction region (1) has a grey background in FIG. 1 and includes the main part of the reaction region (1a), the separate part of the reaction region (1b) and the supplying and removing pipelines.

(Meth)acrylic acid (=(M)AA)) and acetic anhydride (=Ac₂O) are first fed into the main part of the reaction region (1a).

Subsequently, the entire unit is placed under reduced pressure and the reaction mixture is heated with the aid of a heat exchanger (2) in the main part of the reaction region until the rectification column (3) above the main part of the reaction region has filled completely with vapour and liquid.

The heat exchanger (2) is preferably mounted separately and is connected to the main part of the reaction region (1a) via pipelines. The reaction mixture is preferably pumped continuously through the heat exchanger (2) with a pump (4).

The pump to the separate part of the reaction region (5) is now started, and the reaction mixture is also conducted continuously through the separate part of the reaction region (1b).

The separate part is preferably a flow tube reactor which contains an acidic fixed bed catalyst, more preferably an acidic ion exchanger.

The waste stream (6) is recycled back into the main part of the reaction region (1a).

In the rectification column (3), the separation of the components takes place. To prevent polymerization, preference is given to metering in inhibitor at the top of the column.

At the top of the rectification column (7), the acetic acid formed is drawn off first as the lowest-boiling component. Subsequently, the (meth)acrylic anhydride formed is drawn off.

The present invention will be illustrated in detail hereinafter with reference to examples.

### EXAMPLE 1

### Preparation of Methacrylic Anhydride

For the preparation of methacrylic anhydride by reaction of methacrylic acid and acetic anhydride, an experimental plant according to FIG. 1 was constructed.

The rectification column (3) has approx. 25 separating stages. With the top condenser, this column was approx. 3.5 m high, had an internal diameter of 100 mm and was equipped with Sulzer CY structured packings. The polymerization inhibitor used was phenothiazine. The effluents at the top of the column (7) and the heating output of the heat exchanger (2) were controlled by establishing suitable temperatures and internal circulation streams in the experimental plant. The main part of the reaction region (1a) was a glass vessel with a volume of 10 l. The heat exchanger (2) used was a falling-film evaporator.

The experiment was effected both without (Experiment 1.1) and with heterogeneous fixed bed catalyst (Experiment 1.2) in the separate part of the reaction region (1b). The heterogeneous fixed bed catalyst used was 500 ml of the acidic ion exchanger Lewatit K2431 from Lanxess. Before the start of the experiment, the ion exchanger was washed with methacrylic acid in order to flush out the water present in the ion exchanger. The methacrylic acid was subsequently very substantially sucked out of the ion exchanger through a filter.

Methacrylic acid and acetic anhydride were first fed into the main part of the reaction region (1a). Subsequently, the pressure in the unit was reduced to 20 bar at the top of the column and the reaction mixture was conducted continuously through the heat exchanger (2) with the aid of a pump (4) and heated. Once the rectification column had been filled completely with vapour and liquid, the pump (5) which leads to the separate part of the reaction region (1b) and the discharge at the top of the column (7) were started.

The table which follows shows the experimental results obtained in comparison:

| Experiment | | 1.1 | 1.2 |
|---|---|---|---|
| MAA used | mol | 72.92 | 72.92 |
| Ac2O used | mol | 36.46 | 36.46 |
| MAA used | kg | 6.28 | 6.28 |
| Ac2O used | kg | 3.72 | 3.72 |
| Amount of ion exchanger | ml | 0 | 500 |
| Time until the pump is started (0) | h | 1.0 | 1.0 |
| AcOH removal time | h | 8.5 | 2.0 |
| AcOH removal purity | | | |
| Acetic acid | wt % | 74.21 | 93.14 |
| Acetic anhydride | wt % | 6.04 | 1.32 |
| Methacrylic acid | wt % | 13.44 | 3.15 |
| Acetyl methacrylate | wt % | 4.86 | 1.39 |
| Methacrylic anhydride | wt % | 1.44 | 1.00 |
| Amount of AcOH removed | kg | 5.23 | 4.59 |
| MAAH removal time | h | 3.0 | 2.5 |
| MAAH removal purity | | | |
| Acetic acid | wt % | 0.06 | 0.04 |
| Acetic anhydride | wt % | 0.22 | 0.18 |
| Methacrylic acid | wt % | 0.45 | 0.39 |
| Acetyl methacrylate | wt % | 0.80 | 0.23 |
| Methacrylic anhydride | wt % | 96.35 | 98.91 |
| High boilers | wt % | 2.12 | 0.25 |
| Amount of MAAH removed | kg | 4.49 | 5.18 |
| Yield of MAAH removed | % | 79.8 | 92.1 |
| Residue in the reaction region (1) | kg | 0.28 | 0.23 |
| after the experiment has ended | | | |

In the case of use of the ion exchanger (Experiment 1.2),
a) a significantly lower level of by-products (high boilers and acetyl methacrylate) formed,
b) it was possible to reduce the experiment time from a total of 12.5 h to 5.5 h,
c) a lower level of unconverted reactants (Ac2O and MAA) was present in the AcOH removal and MAAH removal,
d) the yield was significantly higher.

### EXAMPLE 2

### Preparation of Acrylic Anhydride

For the preparation of acrylic anhydride by reaction of acrylic acid and acetic anhydride, the same experimental plant as explained in FIG. 1 was used.

The experimental conditions and the procedure were identical to the details given in Example 1.

The experiment was effected both without (Experiment 2.1) and with heterogeneous fixed bed catalyst (Experiment 2.2) in the separate part of the reaction region (1b). The heterogeneous fixed bed catalyst used was 500 ml of the acidic ion exchanger Lewatit K2431 from Lanxess. Before the start of the experiment, the ion exchanger was washed with acrylic acid in order to flush out the water present in the ion exchanger. The acrylic acid was subsequently very substantially sucked out of the ion exchanger through a filter.

The table which follows shows the experimental results obtained in comparison.

| Experiment | | 2.1 | 2.2 |
|---|---|---|---|
| AA used | mol | 81.23 | 81.23 |
| Ac2O used | mol | 40.62 | 40.62 |
| AA used | kg | 5.85 | 5.85 |
| Ac2O used | kg | 4.15 | 4.15 |
| Amount of ion exchanger | ml | 0 | 500 |
| Time until the pump is started (0) | h | 1 | 1 |
| AcOH removal time | h | 10 | 2.25 |
| AcOH removal purity | | | |
| Acetic acid | wt % | 72.15 | 90.03 |
| Acetic anhydride | wt % | 7.71 | 2.81 |
| Acrylic acid | wt % | 13.94 | 4.79 |
| Acetyl acrylate | wt % | 4.86 | 1.32 |
| Acrylic anhydride | wt % | 1.35 | 1.06 |
| Amount of AcOH removed | kg | 5.82 | 5.19 |
| AAH removal time | h | 2.35 | 2.0 |
| AAH removal purity | | | |
| Acetic acid | wt % | 0.07 | 0.03 |
| Acetic anhydride | wt % | 0.25 | 0.23 |
| Acrylic acid | wt % | 0.51 | 0.50 |
| Acetyl acrylate | wt % | 0.97 | 0.37 |
| Acrylic anhydride | wt % | 95.88 | 98.55 |
| High boilers | wt % | 2.32 | 0.32 |
| Amount of AAH removed | kg | 3.92 | 4.58 |
| Yield of AAH removed | % | 76.6 | 89.5 |
| Residue in the reaction region (1) | kg | 0.26 | 0.23 |
| after the experiment has ended | | | |

In the case of use of the ion exchanger (Experiment 2.2),
e) a significantly lower level of by-products (high boilers and acetyl acrylate) formed,
f) it was possible to reduce the experiment time from a total of 13.35 h to 5.25 h,
g) a lower level of unconverted reactants (Ac2O and AA) was present in the AcOH removal and AAH removal,
h) the yield was significantly higher.

1. Improved A process for preparing unsaturated carboxylic anhydrides of the general formula I
R—C(O)—O—C(O)—R (I)
in which R is an unsaturated organic radical having 2 to 12 carbon atoms
by transanhydridizing an aliphatic carboxylic anhydride with a carboxylic acid of the general formula II
R—COOH (II)
in which R is as defined above,
in an apparatus consisting of a reaction region and a rectification column, wherein
a) the reactants are fed into a reaction region in stoichiometric ratios,
b) the carboxylic acid formed as a by-product is removed from the reaction mixture,
c) the carboxylic anhydride of the formula I is subsequently drawn off,
d) the unconverted reactants are recycled into the reaction region,
e) the reaction region comprises a heterogeneous catalyst and
f) one or more polymerization inhibitors are added.

2. The process according to claim 1, wherein a heterogeneous catalyst is present in the entire reaction region or in a portion of the reaction region.

3. The process according to claim 2, wherein an acidic fixed bed catalyst is present.

4. The process according to claim 2, wherein a cationic exchanger is present as the catalyst.

5. The process according to claim 1, wherein the catalyst is present in a separate region of the reaction region.

6. The process according to claim 1, wherein the unsaturated carboxylic anhydride of formula (I) is (meth)acrylic anhydride, prepared by transanhydridization of acetic anhydride and (meth)acrylic acid.

7. A process for preparing unsaturated carboxylic anhydrides of general formula I
R—C(O)—O—C(O)—R (I)
in which R is an unsaturated organic radical having 2 to 12 carbon atoms
by transanhydridizing an aliphatic carboxylic anhydride with a carboxylic acid of general formula II
R—COOH (II)
in which R is as defined above,
in an apparatus comprising a reaction region and a rectification column, wherein
a) the reactants are fed into a reaction region in stoichiometric ratios,
b) the carboxylic acid formed as a by-product is removed from the reaction mixture,
c) the carboxylic anhydride of the formula I is subsequently drawn off,
d) the unconverted reactants are recycled into the reaction region,
e) the reaction region comprises a heterogeneous catalyst and
f) one or more polymerization inhibitors are added.
